(19) United States
(12) Patent Application Publication
(10) Pub. No.: US 2023/0323416 A1
(43) Pub. Date: Oct. 12, 2023

(54) ENZYMATIC DEGRADATION OF CELLULOSIC SUBSTRATES IN THE PRESENCE OF LIGNOCELLULOSE MILLING PARTICLES

(71) Applicant: Xeros Limited, Rotherham, South Yorkshire (GB)

(72) Inventors: Lars Frederik Paul SERVIN, Rotherham (GB); Richard Elliot PALMER-LILLEY, Rotherham (GB); Elizabeth Jean ABERCROMBIE, Glasgow (GB); Stephen Derek JENKINS, Middlesborough (GB)

(21) Appl. No.: 18/023,229

(22) PCT Filed: Aug. 25, 2021
(86) PCT No.: PCT/GB2021/052207

§ 371 Date: Feb. 24, 2023

(30) Foreign Application Priority Data
Aug. 26, 2020 (GB) 2013321.1

(51) Int. Cl. C12P 19/04; C12P 19/02; C12M 1/33; C12M 1/40; C12N 9/42

(52) U.S. Cl. CPC C12P 19/04; C12P 19/02; C12M 45/02; C12M 21/18; C12N 9/2437; C12Y 302/01004

(57) ABSTRACT

A process and apparatus for the enzymatic degradation of a cellulosic substrate is disclosed. The process comprises agitating a composition with milling particles, wherein the milling particles are or comprise a lignocellulosic material and wherein the composition comprises: a. the cellulosic substrate; b. a cellulase enzyme; and c. a liquid medium.

The present invention relates to a process for enzymatic degradation of a cellulosic substrate, and especially to processes for the production of glucose, alcohol, biogases and energy comprising the processes for enzymatic degradation. It further relates to an apparatus for the enzymatic degradation of the cellulosic substrate.

Cellulose-containing materials represent an abundant, low-cost and renewable source for the production of fuels, plastics or chemicals. The use of cellulose-containing materials for the production of fuels in particular is well known. Processes for the production of fuels from cellulose-containing materials typically utilise low-cost waste materials, such as scrap cotton, wheat straw and waste from various industries. Such materials are typically abundant, renewable and may have zero net carbon production.

Such processes typically comprise the conversion of cellulose into sugars using enzyme and/or acid hydrolysis. The sugars are typically fermented to produce alcohols, and the alcohols combusted to produce energy.

Despite the availability and low cost of cellulosic materials, they are often more costly for energy production than fossil fuels. This is partly because of subsidies to the fossil fuel industry, but also because of the large amount of processing and enzymes that cellulosic materials require for conversion into sugars. The processing can also be slow, with some state-of-the-art processes requiring in excess of 72 hours to achieve a viable yield of sugars.

Research has been conducted into ways to increase yield and/or to reduce processing time of the hydrolysis of cellulose into sugars. For example, Kelsey et al. (Enhancement of Cellulose Accessibility and Enzymatic Hydrolysis by Simultaneous Wet Milling; Kelsey et al; 1980; Biotechnology and Bioengineering, Vol. XXVII) proposed the use of steel particles for the ball milling of lignocellulosic substrates in the presence of hydrolysing enzymes. Kelsey el al. reduced processing complexity by combining hydrolysis with milling. However, this process has a number of drawbacks, including the expense of milling with steel balls.

The trend in the art has been to use higher hardness ceramic milling particles to break down the cellulosic feedstock. For example, JP2006-081483 suggests the use of high hardness milling particles in the form of zirconia and alumina along with combined hydrolysis to improve conversion rates. However, ceramic milling particles have an increased cost associated with them. Their use, along with the use of metal milling particles, reduces the economic viability of degrading cellulosic substrates for energy production. Both metal and ceramic milling particles can also wear out the inside surfaces of the ball mill vessel. The wear of such milling particles also creates debris which may need separating from liquid at the end of the ball milling process.

The present invention aims to address any of the problems associated with prior art processes.

In a first aspect of the present invention, there is provided a process for the enzymatic degradation of a cellulosic substrate, the process comprises agitating a composition with milling particles, wherein the milling particles are or comprise a lignocellulosic material, and wherein the composition comprises:
a. the cellulosic substrate;
b. a cellulase enzyme; and
c. a liquid medium.

Aspects and embodiments of the present invention described herein provide an improved process which uses lignocellulosic milling particles. Surprisingly, the present inventors have found that agitating the composition with lignocellulosic milling particles provides improved degradation rates compared to ball milling using steel balls. Furthermore, lignocellulosic milling particles have been found to be more economical and reduce damage to the inside of a milling vessel. The wear debris of lignocellulosic milling particles can also be digested as part of the degradation process or incinerated to provide energy for the process. It was also a surprise to the present inventors that the lignocellulosic milling particles could be re-used many times as it would have been expected that such milling particles would rapidly degrade.

The milling particles comprise a lignocellulosic material. The term “lignocellulosic material” may refer to a material comprising both lignin and cellulose.

Lignin is a polyphenolic material comprised of linked phenyl-propane units. The structure of lignin varies by plant species and location within the plant. Cellulose is a polysaccharide of beta-glucose, comprising glyosidic bonds. The lignocellulosic material may contain hemi cellulose, which is a saccharide that may contain saccharide monomers other than glucose. A lignocellulosic material may for example, comprise between 35-55% cellulose, 15-30% lignin, optionally 23 to 32% hemi cellulose.

The milling particles may be or comprise any one of: wood (especially wood from trees), nutshells, husks, corn cobs, bamboos, fruit stones or any combination thereof. Of these, bamboo is especially preferred. Preferably the milling particles comprise at least 50 wt. % wood, or at least 75 wt. % wood or the milling particle consists entirely of wood.

The milling particles suitably have a density from 300 Kg/m³ to 1400 Kg/m³. Density is measured according to the dry weight of individual milling particles.

The lignocellulosic material of the milling particles suitably have a lignin content from 15 wt. % to 40 wt. %, or from 20 to 30 wt. %, or a range formed from any combination of these endpoints. Lignin content is preferably determined according to the Klason method (the 72% sulfuric acid method). In this method, samples are digested with 72% sulfuric acid, then with dilute sulfuric acid, to hydrolyse and solubilize the polysaccharides; the insoluble residue is dried and weighed as lignin. Acid-soluble lignin from angiosperms can be estimated from the UV absorbance of the hydrolysate.

The milling particles may be or comprise wood. As used herein, the term “wood” may be considered as the lignocellulosic material derived from the stem or from branches of a tree. The term “wood” may include the heartwood, wood and knots of the tree stem. The term “wood” may exclude the bark of the stem or branches of a tree. The term “wood” may also include lignocellulosic material derived from the stem of woody grass species especially wherein the grass is bamboo.

The wood may optionally be or comprise wood from the following exemplary tree species: pine, eucalyptus, poplar, acacia, rubberwood, willow, elm, birch, maple, walnut, cherry, apple, chestnut, beech, oak, hickory, alder, mesquite or the grass species bamboo. In particular, the wood may be or comprise hickory or bamboo and especially bamboo. The wood may derive from hardwood trees and/or softwood trees, or from woody grasses such as bamboo.

Where the milling particles comprise wood, the wood may have a Janka hardness greater than 1000 N, or greater than 1500 N, or greater than 2000 N, or greater than 3000 N, or greater than 4000 N, or greater than 5000 N. The Janka hardness of a wood may be determined in accordance with the ASTM D1037-12 measure for hardness.

The lignocellulosic material of the milling particles may be unrefined. Where the milling particles comprises wood, the wood may be unrefined. As used herein, the term “unrefined” may refer to the lignocellulosic material not being treated by a process to substantially increase the susceptibility of the lignocellulosic material to enzymatic digestion, except for a process necessary to form the lignocellulosic material into milling particles. Processes to form lignocellulosic material into milling particles may include any one of the following: cutting raw material into pieces sized for use as particles (e.g. by sawing, chipping or splitting), seasoning, ageing, drying and/or separating from unwanted material (e.g. removing bark).

The term “unrefined” may exclude any of the following processes: reducing the lignin content or altering lignin structure, removing or altering hemi cellulose, reducing crystallinity or decrystallizing the cellulose of the lignocellulosic material, removing acetyl groups from hemi cellulose, reducing the degree of polymerisation of the cellulose of the lignocellulosic material, increasing pore volume of the lignocellulosic material, and/or increasing the surface area of the lignocellulosic material by any of shredding, grinding or milling.

The lignocellulosic material of the milling particles may be considered to be any material containing lignin and cellulose, where the material is structured so that it is recalcitrant to degradation by a cellulase enzyme.

The milling particles preferably have a size of from 1 mm to 400 mm, or from 10 mm to 200 mm, or from 20 mm to 100 mm, or from 1 mm to 50 mm, or from 15 mm to 40 mm, or from any range formed from a combination of these end points. The size of the milling particle refers to the smallest linear dimension within or across the particle unless otherwise stated herein. The size of a milling particle can be determined using Vernier callipers, for example.

The milling particles may have an angular shape. As used herein, the term “angular shape” may refer to any three-dimensional shape comprising an edge or a vertex. Optionally, the edge may have a maximum angle of 120 degrees of less. The milling particles may have an angular shape that may comprise shapes that are any of: cuboidal, any prism including the prism of a sector, cylindrical, conical, polyhedral and truncated forms of the aforementioned 3D shapes.

The milling particles may have an aspect ratio of from 20:1 to 1:1 or from 10:1 to 2:1, or any range formed from any of these endpoints. The aspect ratio is the ratio of the longest dimension to the shortest dimension measured perpendicular to the longest dimension.

Preferably the longest linear dimension of a milling particle is no more than 400 mm, for any aspect ratio

Where the lignocellulosic material is or comprises wood, the angular shape of the milling particles may comprise forms that typically arise from the processing of wood, these may include shapes that arise from the cutting, splitting or chipping of tree stems, branches or lumber or cutting, splitting or chipping of woody grasses especially bamboo. In particular, the milling particle may be in the form of wood chips.

The milling particles are preferably free from a conventional high hardness milling medium. A conventional high hardness milling medium may comprise metals (a non-limiting example is steel), ceramics (a non-limiting example is alumina), or minerals (non-limiting examples include quartz and silicas). A high hardness material is preferably suitably defined as any material with a Mohs hardness greater than 4, or any material with a Mohs hardness greater than 5.

Preferably, the milling particles comprise no more than 50 wt. %, or no more than 25 wt. %, or no more than 5 wt. %, or no more than 1 wt. % of milling particles which are a high hardness milling media. Preferably, the milling particles comprise no more than 50 wt. %, or no more than 25 wt. %, or no more than 5 wt. %, or no more than 1 wt. % of synthetic polymers.

The composition which is agitated with said milling particles preferably comprises from 1 to 40 wt. % of cellulosic substrate, or from 5 to 30 wt. % of cellulosic substrate, or from 5 to 20 wt. % of cellulosic substrate, or a range formed from any of these endpoints.

The composition preferably comprises from 0.001 to 8 wt. % of cellulase enzymes, or from 0.005 to 4 wt. % of cellulase enzyme, or from 0.01 to 2 wt. % of cellulase enzymes, or any range formed from any of these endpoints. One or more cellulase enzymes may be present in the composition and in the case of more than one cellulase enzyme the amounts in wt % are the total amounts of all cellulase enzymes present in the composition.

The composition preferably comprises from 50 to 99 wt. % of liquid medium, or from 65 to 97 wt. % of liquid medium, or from 80 to 95 wt. % of liquid medium, or any range formed from any of these endpoints.

The composition preferably comprises a ratio of cellulosic substrate to cellulase enzyme of from 1:0.001 to 1:0.5, or a ratio of from 1:0.005 to 1:0.1, or a range of ratios formed from any of these individual ratios. Preferably, the ratio is a weight ratio.

The composition preferably comprises a ratio of cellulosic substrate to liquid medium of from 0.01:1 to 0.5:1, or a ratio of from 0.05:1 to 0.2:1, or a ratio of from 0.075:1 to 0.1:1, or a range of ratios formed from any of these individual ratios. Preferably, the ratio is a weight ratio.

The composition preferably comprises a ratio of milling particles to the composition by weight of from 1:20 to 1:1.05, or a ratio of from 1:10 to 1:1.05, or a ratio of from 1:5 to 1:0.5, or a ratio of from 1:20 to 1:1; or a ratio of from 1:10 to 1:1; or a ratio of from 1:4 to 1:1, or a ratio of from 1:3 to 1:1.5, or a range of ratios formed from any of these individual ratios. A process to the present disclosure may comprise a ratio of milling particles to composition of from 1:5 to 1:1 by weight.

The process may use from 50 to 25,000 litres of the composition, or from 100 to 15,000 litres, or from 1,000 to 10,000 litres, or a range formed from any of these endpoints.

The composition may additionally comprise one or more of: buffering agents, acids or non-cellulase enzymes.

The composition suitably has a pH of from 4 to 8, or from 5 to 7, or any range formed from any of these endpoints.

Non-limiting examples of buffering agents and acids may include one or more of: sodium citrate, sodium acetate, sodium phosphate, potassium phosphate, citric acid, acetic acid, formic acid. The composition may comprise from 1 to 2 wt. % of buffering agents and/or acids.

Non-limiting examples of non-cellulase enzymes may include hemi cellulases (e.g. xylanase, β-xylosidase, glucuronidase, acetylesterase, galactomannanase, glucomannanase), amylases, ligninases and pectinases. The composition may comprise from 0.005 to 1 wt. % of non-cellulase enzymes.

The cellulosic substrate may be or comprise a waste feedstock. The term “waste feedstock” may refer to a by-product from an industrial process or discarded material. Discarded material may include feedstocks which have come to the end of their serviceable life for example cotton garments which are faded, torn, stained or worn or feedstocks which have been used once, examples of which include recycled paper. A waste feedstock may include herbaceous waste, agricultural residue, forestry residue, municipal solid wastes, wastepaper, waste fabrics or fibres, animal faeces, pulp and paper mill residues, or a combination thereof.

The term “cellulosic substrate” refers to a substrate containing cellulose, the term does not exclude that the substrate additionally comprises lignin.

The cellulosic substrate may be or comprise one or more cellulose-containing fibres, including any of cotton, flax, rayon, bamboo, sugarcane, sisal, abaca, jute, kenaf, banana, capok, coir, pina, raffia, ramie, hemp or a mixture thereof. Bamboo is less preferred as a cellulosic substrate.

The cellulosic substrate may be or comprise paper, cardboard, pulp or a mixture thereof.

The cellulosic substrate may be or comprise leaves, plant stalks, roots, straw, stover and/or grasses.

The cellulosic substrate may comprise bagasse, miscanthus, sorghum residue, plant husks, plant roots, leaves or grasses (including or excluding those from bamboo). Non-limiting examples of husks include, barley husks, wheat husks, rye husks, rice husks, millet husks, sorghum husks, corn husks, rapeseed husks, cotton seed husks & sunflower seed husks.

The cellulosic substrate may consist entirely of any one of the aforementioned cellulosic substrates.

The cellulosic substrate may have been refined prior to use. The term “refined” or “refining” in this context may refer to any process or processes to increase enzymatic digestibility. Such processes may include one or more of: reducing the lignin content or chemically altering the lignin, removing or altering hemi cellulose, reducing crystallinity or decrystallizing cellulose, removing acetyl groups from hemi cellulose, reducing the degree of polymerisation of cellulose, increasing pore volume of the cellulosic substrate, or substantially increasing the surface area of the cellulosic substrate by grinding or milling.

Refining may be performed by applying one or more of the following exemplary processes: autohydrolysis, steam treatments (including steam explosion), grinding, milling, radiation (including microwave treatments), flow through liquid hot water treatments, ammonia fibre expansion (AFEX), hydrothermal treatments, biological treatments, catalytic treatments, non-catalytic treatments, acid treatments, supercritical carbon dioxide treatments, alkali treatments (including treatment with any of sodium hydroxide, lime, ammonia and oxidative alkalis), organic solvent treatments, non-cellulase enzymatic treatments, cellulose solvent treatments, and treatments with aerobic fungi.

The term “refining” in this context may exclude processes which do not substantially increase the susceptibility of a substrate to enzymatic digestion. In particular, “refining” may exclude any of cutting raw material sized for use as milling particles (e.g. by sawing, chipping or splitting), seasoning, ageing, drying and/or separating from unwanted material (e.g. removing bark).

The cellulosic substrate is preferably different from the milling particle. Autologous milling, whereby agitation of a substrate is considered to mill itself, is therefore preferably excluded from the first aspect. The cellulosic substrate is preferably compositionally different from the milling particle or has undergone a refining step to make it different to the milling particle.

In particular, the cellulosic substrate may be a substrate in a form where the cellulose component can be readily degraded by a cellulase enzyme. For example, untreated wood does not meet this preference as the structure, as well as the crystalline cellulose, lignin and hemi cellulose forms, prevent substantial degradation by cellulase enzymes. A readily degraded cellulosic substrate may be one where the yield of saccharides in the presence of an aqueous medium containing 0.5 wt. % endo-cellulase exceeds 1% in a period of 6 hours, at optimal pH and temperature of the endo-cellulase.

The liquid medium is preferably aqueous. The liquid medium may comprise at least 90 wt. % of water, or at least 95 wt. % of water.

The cellulase enzyme may comprise one or more enzymes capable of degradation of cellulose.

The cellulase enzyme may be or comprise an endo-cellulase. Endo-cellulases are cellulases that cleave polysaccharide polymer chains internally by breaking 1,4-β-D-glycosidic bonds in the cellulose backbone.

The cellulase enzyme may be or comprise an exo-cellulase (also referred to as exoglucanases or cellobiohydrolases). Exo-cellulases are cellulases that cleave cellobiose from the reducing and non-reducing ends of cellulose and molecules generated by the action of endo-cellulases.

The cellulase enzyme may include any of, and preferably at least one each of, cellobiohydrolases, endoglucanases and beta-glucosidases.

In particular, the cellulase enzyme may comprise an enzyme that catalyses the hydrolysis of 1,4-β-D-glycosidic linkages. This may include commercially available cellulases including but not limited to: CELLUSOFT™, CELLUCLAST™, CELLUZYME™, CEREFLO™, ULTRAFLO™, CELLIC CTec2® (all available from Novozymes A/S), ACCELLERASE™, SPEZYME™ CE, SPEZYME™ CP (available from IFF-DuPont Nutrition & Biosciences.) and ROHAMENT® CL (from AB Enzymes GmbH).

Agitation of the composition may be performed for a period of from 1 minute to 96 hours, or for a period of from 1 minute to 72 hours, or for a period of from 6 hours to 72 hours, or for a period of 2 hours to 12 hours or for a period from 12 hours to 48 hours, or for a period of 18 to 36 hours, or for a period from 12 hours to 24 hours, or for any range defined by any of these endpoints. A process according to the present disclosure may be comprise the agitation performed for a period of from 12 hours to 48 hours.

Agitation of the composition may be performed in a milling vessel. The milling vessel may be cylindrical in shape. Agitation of the composition may be performed by rotation of the milling vessel. The milling vessel may have an axis extending through the axis of rotational symmetry of the vessel. The longest dimension of the milling vessel may also be aligned with this axis. The milling vessel may optionally be aligned with this axis in the horizontal direction. Agitation may alternatively be performed by a stirrer located within the milling vessel, by oscillation of the milling vessel, or by ultrasound emitter.

Agitation can be provided by rotation (especially tumbling), shaking, oscillating, vibrating, ultrasonication or any combination thereof of the composition and the milling particles.

Where agitation is performed by rotation of a milling vessel, the speed of rotation of the vessel may be selected so that the fall height of the milling particles in the milling vessel is maximised. Rotation of the milling vessel may therefore induce a centripetal force of from 0.2 to 1.0 G, or from 0.3 to 0.9 G, or from 0.4 to 0.8 G, or from 0.5 to 0.7 G, or in a range formed from any of these endpoints. The centripetal force is preferably measured or calculated from the inner surface of the walls of the milling vessel. Preferably, the relevant surface is that furthest from the axis of rotation of the milling vessel or of the stirrer.

The composition preferably has a temperature of from 20 to 60° C., or from 30 to 50° C. during agitation.

After agitation, the milling particles are preferably separated from the composition. This may be the first separation step. Separation may be performed by filtering through a porous material (e.g. a mesh). The pore size of the porous material may be sized to retain the milling particles and allowing passage of the composition. The pore size of the porous material may be selected to permit passage of particles of undegraded or partially degraded substrate. The pore size of the porous material may additionally or alternatively be selected to allow passage of fragments of milling particle. The pore size of the porous material may be from 1 to 400 mm, or from 10 mm to 200 mm, or from 20 mm to 100 mm, or in a range formed from any of the preceding endpoints. The term pore size used herein may refer to the largest linear size of a pore aperture.

The porous material may comprise a mesh or perforated sheet, although other porous materials may be used. The porous material may be formed of a polymer or a metallic material. The porous material may optionally be integrated into an outlet of the milling vessel.

The composition once separated from the milling particles may be subject to further saccharification or may be subject to any other processes as disclosed herein.

A second separation step may optionally be performed on the filtered composition. The second separation step may be performed to remove any of crystalline cellulose, hemi cellulose, lignin, fragments of the milling particles, or any other non-liquid components of the degraded cellulosic substrate. The second separation step may comprise filtering with a second porous material, wherein the pore size of the second porous material is smaller than the pore size of the porous material used as the filter in the first separation step. The second separation step may optionally or alternatively comprise separation using a cyclonic or centrifugal separation apparatus or by membrane separation. Where membrane separation is used, the membrane may be configured to extract saccharides from the composition and to retain enzymes in the composition for re-use.

Some or all of the milling particles may be re-used as the milling particles in a subsequently performed process for the enzymatic degradation of a cellulosic substrate according to the first aspect of the present invention. If the milling particles are removed from a milling vessel after agitation, they may be returned to the same or another milling vessel for re-use. Alternatively, if the milling particles are retained in a milling vessel after agitation, the milling particles may be re-used by adding fresh composition and repeating the process according to the first aspect.

The re-use of the milling particles in a subsequent process for the degradation of the cellulosic substrate may be performed with the addition of virgin milling particles to the re-used milling particles. Optionally, virgin milling particles may be added in an amount of from 0.1 to 75%, or from 1 to 50% by weight of the total milling particle weight.

The milling particles are typically re-used in no greater than 100, 50, 20, or 10, or 5 iterations of a process according to the first aspect. The milling particles are preferably re-used in at least 1 or 2 iterations of a process according to the first aspect.

The milling particles may decrease in dry mass after the first or any subsequent iteration of the process according to the first aspect. The decrease in mass after the first or any subsequent iteration may be from 0 to 5%, or from 0.5 to 4%, or from 1 to 3% or in a range formed from any combination of these endpoints. These decreases in mass are typically measured after the 5th iteration of the process according to the first aspect of the present invention.

Used milling particles may be incinerated to provide energy for the process according to the present disclosure, optionally the energy may be provided in the form of steam. Additionally, lignin and wear debris from the milling particles may also be incinerated.

Used milling particles may be converted to a cellulosic substrate after one or more iterations of the process according to the first aspect. The conversion of the milling particles to a cellulosic substrate may allow the production of saccharides, glucose or alcohols from waste milling particles. In particular, conversion may be performed if the milling particles have been degraded to an extent where their efficacy as a milling particle is much reduced or is no longer viable.

Conversion of the milling particles to a cellulosic substrate may comprise any refining process to increase enzymatic digestibility. This may include one or more of: reducing lignin content or altering lignin composition, removing or altering hemi cellulose, reducing crystallinity or decrystallizing cellulose, removing acetyl groups from hemi cellulose, reducing the degree of polymerisation of cellulose, increasing pore volume of the material, or substantially increasing the surface area of the material by grinding or milling.

Conversion of the milling particle to a cellulosic substrate may be performed by applying one or more of the following exemplary processes: autohydrolysis, steam treatments (including steam explosion), grinding, milling, radiation (including microwave treatments), flow through liquid hot water treatment, ammonia fibre expansion (AFEX), hydrothermal treatments, biological treatment, catalytic treatment, non-catalytic treatments, acid treatments, supercritical carbon dioxide treatments, alkali treatments (including treatments with any of sodium hydroxide, lime, ammonia and oxidative alkalis), organic solvent treatments, non-cellulase enzymatic treatments, cellulose solvent treatments, and treatments with an aerobic fungi.

Enzymatic degradation of the cellulosic substrate with a cellulase enzyme may produce a composition comprising monosaccharides and/or oligosaccharides. It will be appreciated that the monosaccharides and/or oligosaccharides are produced from the cellulose in the cellulosic substrate.

After the process of the first aspect has completed, from 10 to 90% of the cellulose in the cellulosic substrate has typically been converted to monosaccharides and/or oligosaccharides by the cellulase enzymes.

The process of the first aspect may comprise the step of separating the monosaccharides and/or oligosaccharides from the composition after agitation. This may be achieved by filtration to remove the milling particles and remaining cellulosic substrate. The liquid medium may then optionally be removed by drying, for example vacuum drying or freeze drying.

The process according to the first aspect may be performed as a continuous, batch or fed-batch process.

In a second aspect of the invention there is provided a process for the production of glucose from a cellulosic substrate, comprising the process according to the first aspect to produce monosaccharides and/or oligosaccharides, and further comprising the conversion of said monosaccharides and/or oligosaccharides to glucose by further enzymatic degradation (also referred to herein as digestion). Thus, the second aspect produces a composition comprising glucose. Preferably, this process produces oligosaccharides which are converted to glucose by further enzymatic degradation.

Further enzymatic degradation may be performed by exposing the composition comprising monosaccharides and/or oligosaccharides to a further enzyme. The further enzyme suitably comprises one or more of: ligninases, hemi cellulases (e.g. xylanase) or further cellulases (e.g. β-glucosidase).

The composition comprising monosaccharides and/or oligosaccharides may be transferred from the milling vessel to a separate vessel and combined with the further enzyme or enzymes. The composition comprising monosaccharides and/or oligosaccharides may be combined with the further enzymes for a period of at least 6 hours, 12 hours or 24 hours, and/or no more than 12 hours, 24 hours or 48 hours.

The composition comprising monosaccharides and/or oligosaccharides may undergo one or more filtration processes to remove from the milling particles, fragments of the milling particles, fragments of undegraded cellulose substrate, crystalline cellulose and lignin. Filtration may be performed by any separation process disclosed herein. The composition may also be filtered using membrane separation to remove saccharides for re-use of the enzymes.

The composition comprising monosaccharides and/or oligosaccharides may be thickened or dewatered prior to adding the further enzyme or enzymes.

The process according to the second aspect may optionally be performed in the milling vessel and may optionally be performed simultaneously with, or after the process of the first aspect.

The process according to the second aspect may be performed as a continuous, batch or fed-batch process.

In a third aspect of the invention, there is provided a process for the production of one or more alcohols from a cellulosic substrate, comprising the process according to the second aspect, and fermenting the glucose to produce an alcohol-containing composition.

The fermenting may comprise the use of microorganisms. The microorganisms in particular may include yeast. Prior to fermentation, the glucose-containing composition from the second aspect may be dewatered, filtered or the glucose may be separated therefrom. After fermentation the alcohol-containing composition may be distilled to extract the one or more alcohols from the composition.

Alternatively, glucose or the glucose-containing composition may be used as a feedstock for yeast, bacteria or other microorganisms that can be used in the production of enzymes, therapeutics, bio-derived polymers and other microorganism-derived molecules.

In a fourth aspect, one or more biogases may be produced by anaerobic digestion of the monosaccharides (e.g. glucose) and/or oligosaccharides. Preferably, the biogases comprise methane. Other gases which may be present may include carbon dioxide and optionally hydrogen sulfide, moisture and siloxanes. Preferably, the biogases comprise at least 10%, at least 20%, at least 30% or at least 40% methane (all being percentages by volume).

The process according to the third aspect may be performed as a continuous, batch or fed-batch process.

The process according to the third aspect may be performed on a composition containing at least 8 w/w % of fermentable sugars and may produce a composition containing at least 4 w/w % of one or more alcohols. Such compositions may optionally have been derived from a composition comprising at least 15 wt. % of cellulosic substrate.

In a fifth aspect of the invention, there is provided a process for the production of energy from a cellulosic substrate, comprising a process according to the third aspect or fourth aspect wherein the one or more alcohols or biogases are combusted to release energy. Energy may be used to generate steam or gases to drive an electrical generator or to drive other mechanical apparatus.

In a sixth aspect of the invention there is provided an apparatus for the enzymatic degradation of a cellulosic substrate, the apparatus comprising a milling vessel charged with a composition and milling particles, wherein the milling particles are or comprise a lignocellulosic material; and wherein the composition comprises the cellulosic substrate, a cellulase enzyme and a liquid medium. The apparatus may be used to perform any of the processes of the first aspect.

The milling vessel may comprise a rotary ball milling vessel, where the vessel is rotated once the vessel is charged with a milling particle. Alternatively, the vessel may comprise a stirred ball milling vessel, where a stirrer is rotated in the vessel when charged with a milling particle. The milling vessel may be adapted to perform wet milling (i.e. the milling of a substrate in the presence of a liquid medium).

The milling vessel may have a capacity of no less than 10 L; 100 L; 1,000 L; 10,000 L or 100,000 L. The milling vessel may have a capacity no greater than 50 L; 500 L; 5,000 L; 50,000 L; 500,000 L or 1,000,000 L.

The milling vessel may have a hollow form of a generally cylindrical, cuboidal, or regular prism shape. The milling vessel may comprise a long axis, the long axis may extend through centre of rotational symmetry of the generalised shape of the milling vessel. The long axis of the milling vessel may be arranged horizontally or vertically. Where the vessel is a rotary ball mill vessel, the vessel may be rotated along its long axis. In particular the milling vessel may comprise a hollow elongate cylinder rotatable about the long axis with the long axis aligned in the horizontal direction.

The milling vessel may comprise one or more closable opening(s) sized for the charging of the milling vessel with the composition and the milling particles and/or for the removal of processed composition and optionally milling particles. The milling vessel may comprise a porous material for the separation of milling particles from the composition. The porous material may be removably comprised in the closable opening or may be integrated into an additional opening.

The milling vessel may comprise or be attached to an apparatus for membrane separation. The membrane separation apparatus may be configured to extract saccharides from the composition. Separation may be performed during or after a process according to the first aspect. The membrane separation apparatus may be configured to facilitate the removal of the saccharides from the milling vessel whilst retaining enzymes in the vessel for subsequent re-use. The membrane separation apparatus may be located internally to the vessel or externally therefrom. Where the membrane separation apparatus is located externally, the composition may be removed from the milling vessel and passed through the membrane separation apparatus, then optionally returned to the milling vessel.

The opening(s) of the milling vessel may be adapted for batch processes, fed-batch processes and continuous processes.

The milling vessel may comprise one or more sensors to determine any one or more of the following: temperature, pH or concentration of glucose, other saccharides or enzymes.

The milling vessel may comprise heating and/or cooling apparatus. Heating and cooling apparatus may comprise, amongst others, conduits for the passage of hot or cold fluids within the milling vessel. Heating apparatus may comprise heating elements internally or externally of the milling vessel.

The milling vessel may comprise a controller to control agitation of the composition in the milling vessel. The controller may be configured to agitate the composition at different intensities throughout the process according to the first aspect. The controller may be configured to agitate at a high intensity for a first period to mill the cellulosic substrate in the composition. The controller may be configured to agitate at a low intensity for a second period to stir the composition with minimal milling of the cellulosic substrate.

The milling vessel may be comprised of metals or alloys such as steel, or any other material suitable for the construction of a vessel. Lignocellulosic materials are less abrasive and also less dense than steel or ceramic abrasive particles. The milling vessel can therefore be made to a lower specification than a milling vessel designed for steel or ceramic milling particles, reducing the capital expenditure.

It will be appreciated that the features, preferences and embodiments described hereinabove may be applicable where combinations allow, to each of the figures. Embodiments of the invention are further described with reference to the following figures.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise. By way of examples a cellulosic substrate means one or more cellulosic substrates and a cellulase enzyme means one or more cellulase enzymes.

Features, integers, characteristics, compounds, chemical moieties or groups described in conjunction with a particular aspect, embodiment or example of the invention are to be understood to be applicable to any other aspect, embodiment or example described herein unless incompatible therewith. Thus, it will be appreciated that the features and preferences of the first aspect of the invention are also applicable to the second, third, fourth, fifth and sixth aspects of the invention. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

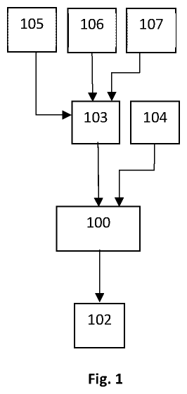

FIG. 1 is a block diagram showing a process for the degradation of a cellulosic substrate according to the first aspect.

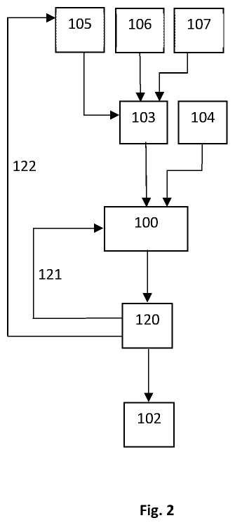

FIG. 2 is a block diagram showing a process for the degradation of a cellulosic substrate according to an embodiment of the first aspect.

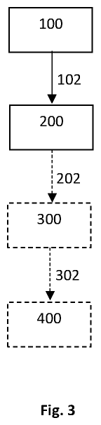

FIG. 3 is a block diagram illustrating processes according to the second, third and fifth aspects.

FIG. 1 illustrates a process 100 according to the first aspect of the invention. In FIG. 1, a cellulosic substrate 105 is comprised as part of a composition 103. The composition additionally comprises one or more cellulase enzymes 106 and a liquid medium 107. The cellulosic substrate is degraded by agitating the composition in the presence of lignocellulosic milling particles 104, which is or comprises a lignocellulosic material.

In an embodiment, agitation may be performed by charging a milling vessel with the composition 103 and lignocellulosic milling particles 104. The milling vessel may be a 40 m² cylindrical rotary milling vessel configured to rotate around an axis of rotation aligned in the horizontal direction. The composition 103 may comprise 10,000 L of water 107, 10 Kg of enzymes 106 and 1,000 Kg of cellulosic substrate 105. The milling particles 104 may be 5,000 Kg of untreated and unrefined wood. The milling vessel may be rotated for a duration of 24 h and its contents may be maintained at a temperature of 45° C.

Agitation of the composition 103 in the presence of the milling particles 104 mechanically breaks up the cellulosic substrate 105 and causes the mechanical breakdown of, amongst others, crystalline cellulose, hemi cellulose and lignin components that may be present in the cellulosic substrate 105. This increases the surface area of the cellulosic substrate 105 and increases degradation of the cellulose in the substrate. Agitation of the composition 103 in the presence of the milling particles 104 also increases mixing of the enzymes 106 with the cellulosic substrate 105, the mechanical action of the agitation can drive the enzymes into the pores of the cellulosic substrate 105, further enhancing the rate of degradation. The degradation of the cellulosic substrate 105, by process 100 results in a degraded composition 102 which includes sugars produced by the enzymatic degradation of the cellulose in the cellulosic substrate 105. The degraded composition may also comprise lignin, hemi celluloses and undigested crystalline cellulose.

The milling particles 104 comprise a lignocellulosic material. During process 100, the agitation may cause small fragments to be broken off the milling particles. These fragments may additionally be mechanically degraded by the milling particles and the fragments enzymatically degraded by the cellulase enzymes. This degradation may produce sugars, lignin, hemi celluloses and other products similar to the digestion of the cellulosic substrate 105. Beneficially, the use of a lignocellulosic milling particles 104 removes the need for the fragments of the milling particles to be separated from the degraded composition 102 after degradation by process 100. Additionally, lignocellulosic milling particles 104 are cheaper than conventional milling particles such as alumina or steel balls, advantageously cause less wear of milling vessels, and have been demonstrated to be surprisingly more effective. Lignocellulosic milling particles are also lower in density and, advantageously, agitation with lignocellulosic milling particles uses less energy.

An embodiment of process 100 is shown in FIG. 2, wherein process 100 is followed by separation 120 of the lignocellulosic milling particles 104 from the degraded composition 102. Separation may be performed by filtering with a porous material (i.e. a mesh) for example. The separated lignocellulosic milling particles 104 may be reused 121 as the milling particle 104 in a subsequent iteration of process 100 with new cellulosic feedstock. Lignocellulosic material may be inherently porous and may have a considerably higher surface area compared to conventional milling particles. A significant quantity of enzymes 106 can become adsorbed onto the surface of the lignocellulosic milling particles 104; thus, re-use of lignocellulosic milling particles 104 can cause the re-use of a portion of the enzymes 106, thereby advantageously reducing the quantity of enzymes 106 that need to be added in further iterations of process 100.

After a plurality of re-uses 121 as a milling particle, the milling particles may be discarded, or may undergo a pre-treatment/refinement step 122 and may be used as a cellulosic substrate 105 for agitation with new milling particles 104 as part of another iteration of process 100. Thus, at end-of-life, no waste milling particles are generated.

FIG. 3 illustrates processes according to the second, third and fifth aspects of the invention. As explained above and illustrated in FIGS. 1 and 2, a process 100 for the degradation of a cellulosic substrate involves the agitation of a composition in the presence of lignocellulosic milling particles. The enzymatic degradation of the cellulose in the cellulosic substrate by the enzyme(s) in the composition results in the production of saccharides. In process 200 the saccharides are converted to glucose by further enzymatic degradation. As part of process 200, degraded composition 102 may be exposed to enzymes such as β-glucosidase, hemi cellulases or further cellulases for a period of time of from 1 to 6 hours. This may yield a composition 202 with a typical glucose content of from 1 to 9%. Thus, a second aspect comprises processes 100 and 200.

The glucose-containing composition 202 may undergo a subsequent process of fermentation 300 by a microorganism such as yeast. Fermentation 300 may convert some of the glucose and optionally some of the saccharides in the glucose-containing composition 202 to an alcohol-containing composition 302. Thus, a third aspect comprises processes 100, 200 and 300.

The alcohol may be distilled from the alcohol-containing composition 302 and may be combusted 400 to produce energy which may be in the form of heat and/or which may be converted to motion or electrical energy. Thus, a fifth aspect comprises processes 100, 200, 300 and 400.

### Experimental Data

### Milling Particles

Bar-be-quick® (Rectella International Ltd, Burnley, UK) hickory smoking wood chips of size between approximately 2 mm (shortest dimension) and approximately 4 cm (longest dimension) were used as the lignocellulosic (wood) milling particles. For each test, a dry weight of 145.6 g wood milling particles were first soaked in cold tap water in a static beaker overnight. On the morning of the test, wood milling particles were strained in a coarse sieve and remaining non-absorbed water was removed using a paper towel. Between consecutive interations, the wood chips were thoroughly rinsed with tap water to remove enzyme and non-degraded cotton particles.

Spherical stainless steel (SS) ball bearings (milling particles) of size 3.5 mm and 1.3 cm size were used for the comparative tests in an amount of 145.6 g when dry.

### Cellulosic Substrate

White woven mercerised cotton sheet fabric (Whaleys Bradford Ltd, Bradford, UK) was used as the cellulosic substrate in the tests. Cotton sheet fabric was cut into small pieces of 1 cm in size.

### Enzyme

The cellulase enzyme used in the tests was Cellusoft® LT 19500 L (Novozymes A/S, Bagsværd, Denmark). This enzyme product was determined to be a cellulase blend containing at least endoglucanase and β-glucosidase activities, as it demonstrated activity in the conversion of cellobiose to glucose.

### Apparatus

The stirring device used was a Hei-Torque 400 overhead stirrer (Heidolph, Schwabach, Germany), which was held in a 90° horizontal axial position using a fixed frame and weight balancing. The milling vessel used was a 1.125 litre stainless steel ball mill pot (Capco, a division of Castle Broom Engineering Ltd, Ipswich, UK). The ball mill pot was modified by welding one stainless steel lifter of 2 cm height, and a nut in the centre of the base for connection to the stirring rod of the stirring device, so that rotation of the stirring device rotated the steel ball mill pot. The heating was provided by a Mini Kitchen fan assisted convection oven (Russell Hobbs, Failsworth, UK). The heating temperature setting was calibrated to maintain 340 ml of water at a temperature of 40±2° C. during 24 hours of rotation.

### Combined Milling and Enzymatic Degradation

The stainless steel ball mill pot was first pre-heated in the oven for at least 2 hours, and at least 6 hours with the stainless steel milling particles. The process water as the liquid medium was buffered at pH 6 using 50 mM sodium citrate and citric acid, and pre-heated to around 40° C. using a hotplate. The pre-heated stainless steel ball mill pot was loaded with the milling particles, 340 ml of pre-heated buffer, 6.8 g (20 g/L) of cotton pieces, and 0.408 ml (6% by weight of substrate) of Cellusoft® LT 19500 L. The ball mill pot was then rotated at 83 rpm (equating to a centripetal force of 0.45 G on the inner surface of the milling vessel) for 24 hours and samples taken at the specified time points. Samples were heated at 80° C. for 15 minutes to denature the enzymes and stop the reaction.

In order to account for any non-specific background absorbance from either slight degradation of the wood chips, or the release of reducing substances from the wood chips, consecutive wood chips (no substrate) control tests were also performed.

A Brennenstuhl® PM 231 E wattmeter was connected to a multi-socket adaptor feeding both the stirrer and the oven, and the energy consumption was measured after 24 hours in kilowatt hours (kW.h) to one decimal place.

### Oligosaccharide and Monosaccharide Concentration Determination

### Dinitrosalicylic Acid Assay Method

Oligosaccharide and monosaccharide concentration was determined using the dinitrosalicylic acid (DNSA) assay. Solution part ‘a’ was prepared by dissolving 75 g sodium potassium tartrate in 125 ml deionized water. Solution part ‘b’ was prepared by dissolving 2.5 g of 3,5-dinitrosalicylic acid in 50 ml of 2 N NaOH solution. DNSA reagent was prepared by mixing solution parts a & b and raising the final volume to 250 ml with deionized water. The reagent was stored in a brown glass jar in a refrigerator. A calibration curve was prepared using a concentration range of pure cellobiose (0, 0.25, 0.5, 1, 2, 5, 10 and 20 g/L). To carry out the assay, 2 ml of DNSA reagent was added to 1 ml of each sample, shaken, and vials were placed in a bath of boiling water for 5 minutes to develop colour. Vials were then transferred into a bath of ice-cold water for 10 minutes to quench the colour change reaction and topped up with 9 ml of deionized water. UV-visible light transmission through the samples was measured in a quartz cuvette at 540 nm using a Konica-Minolta CM-3600A spectrophotometer.

### Data Analysis

Percentage transmission (%T) values were converted into absorbance units using the equation Absorbance=2-LOG(%T). Absorbance values for buffer only (blank) and the appropriate consecutive wood chips (no substrate) control test samples were subtracted to account for non-specific background absorbance. Oligosaccharide and monosaccharide concentration was then determined using the gradient equation of the linear cellobiose calibration curve (0-20 g/L). To calculate the % conversion of cellulose to saccharides, the saccharide concentration was divided by the maximum theoretical saccharide release (19 g/L, assuming a 95% cellulose content of the cotton substrate) and multiplied by 100.

### EXAMPLE 1

Table 1 shows the saccharide concentration in g/L at various time points during combined milling and enzyme degradation of cotton using an equivalent dry weight (145.6 g) of wood or comparative (stainless steel) milling particles.

TABLE 1
Oligosaccharide and monosaccharide release using
an equivalent weight of milling particles

| Time, | No milling | 3.5 mm SS | Wood |
|---|---|---|---|
| h | particles | balls | chips |
| 2 | 0.04 | 0.61 | 0.65 |
| 4 | 0.20 | 1.46 | 1.66 |
| 6 | 0.32 | 2.07 | 2.22 |
| 24 | 1.32 | 5.30 | 6.46 |

This shows superior yeild of saccharides from the use of the wood chips as a milling particles compared to an equivalent weight of stainless steel balls.

Table 2 shows the % conversion of cellulose to oligosaccharides and monosaccharides at various time points during combined milling and enzyme degradation of cotton using an equivalent dry weight (145.6 g) of wood or comparative (stainless steel) milling particles.

TABLE 2
percent conversion of cellulose using an
equivalent weight of milling particles

| Time, | No milling | 3.5 mm SS | Wood |
|---|---|---|---|
| h | particles | balls | chips |
| 2 | 0.2 | 3.2 | 3.4 |
| 4 | 1.1 | 7.7 | 8.7 |
| 6 | 1.7 | 10.9 | 11.7 |
| 24 | 7.0 | 27.9 | 34.0 |

This shows superior conversion of cellulose to oligosaccharides and monosaccharides from the use of the wood chips as a milling particles compared to an equivalent weight of stainless steel balls.

### EXAMPLE 2

Table 3 shows both the saccharide concentration and the % conversion of cellulose to oligosaccharides and monosaccharides after 24 hours of combined milling and enzyme degradation of cotton using an equivalent volume (35%) of wood or comparative (stainless steel) milling particles. After five consecutive uses and air-drying for 48 hours, the dry weight of the wood chips was 141 g, indicating a weight loss of 3%.

TABLE 3
Oligosaccharide and monosaccharide release and % cellulose
conversion using an equivalent volume of milling particles

| | No | 1.3 cm | Wood | Wood | Wood | Wood | Wood | Wood |
|---|---|---|---|---|---|---|---|---|
| | milling | SS | chips | chips | chips | chips | chips | chips, |
| | particles | balls | 1 | 2 | 3 | 4 | 5 | mean |
| Saccharide | 1.32 | 4.85 | 6.46 | 5.83 | 6.15 | 5.86 | 6.55 | 6.17 |
| release, g/L | | | | | | | | |
| Conversion, | 7.0 | 25.5 | 34.0 | 30.7 | 32.3 | 30.9 | 34.5 | 32.5 |
| % | | | | | | | | |

This shows superior yeild of saccharides and superior conversion of cellulose to saccharides from the use of the wood chips as a milling particles compared to an equivalent volume of stainless steel balls. The minimal weight loss of the milling particles also demonstrates that the increase of yield and conversion is due to improved milling rather than the lignocellulose in the milling particle being itself converted to saccharides.

Table 4 shows the combined energy consumption in kilowatt hours (kW.h) for the heating and the horizontal axis rotation of the steel milling vessel after 24 hours of combined milling and enzyme degradation of cotton using an equivalent volume (35%) of wood or comparative (stainless steel) milling particles.

TABLE 4
Energy consumption using an equivalent
volume of milling particles

| | Energy consumption, | Energy consumption/% |
|---|---|---|
| | kW · h | cellulose conversion |
| 1.3 cm SS balls | 1.5 | 0.056 |
| Wood chips 1 | 1.3 | 0.038 |
| Wood chips 2 | 1.3 | 0.042 |
| Wood chips 3 | 1.4 | 0.043 |
| Wood chips 4 | 1.4 | 0.045 |
| Wood chips 5 | 1.3 | 0.038 |
| Wood chips, mean | 1.3 | 0.040 |

This shows a reduction in energy consumption from milling with a lignocellulosic milling particle compared to stainless steel balls.

### EXAMPLE 3

### Milling Particles

Natural bamboo circles were obtained from Bakerross.co.uk and sawn into pieces between approximately 2 cm (shortest dimension) and approximately 3 cm (longest dimension). These were used as the lignocellulosic milling particles. In order to prevent any background interference in the DNSA assay from the release of reducing substances from the bamboo pieces, they were first subjected to multiple days of washing by tumbling without enzyme or substrate. Samples were subjected to the DNSA assay to confirm that no more reducing substances were being released. For each test, a dry weight of 400 g bamboo milling particles were first soaked in cold tap water in a static beaker overnight. When stainless steel milling particles were used in comparative tests the amount used was the same volume as the bamboo milling particles. On the morning of the test, bamboo milling particles were strained in a coarse sieve and remaining non-absorbed water was removed using paper towel. In-between consecutive cycles, the bamboo milling particles were thoroughly rinsed with tap water to remove enzyme and non-degraded cotton particles. The bamboo milling particles were tested using the same methodology as for Examples 1 and 2.

Table 5 shows the reducing sugar concentration in g/L at various time points during combined milling and enzyme hydrolysis of cotton using an equivalent volume (35%) of bamboo pieces, wood chips or comparative (stainless steel) milling particles.

TABLE 5
Reducing sugar release using an equivalent
volume of milling particles

| Time, | No milling | 1.3 cm SS | Wood | Bamboo |
|---|---|---|---|---|
| h | particles | balls | chips | pieces |
| 2 | 0.04 | 1.42 | 0.65 | 0.86 |
| 4 | 0.20 | 2.03 | 1.66 | 1.54 |
| 6 | 0.32 | 2.91 | 2.22 | 2.30 |
| 24 | 1.32 | 4.85 | 6.46 | 6.21 |

This shows a superior yield of sugar after 24 hours from the use of the bamboo pieces as milling particles compared to an equivalent volume of stainless steel balls, and that the performance is similar to wood chips.

Table 6 shows the % conversion of cellulose to reducing sugars at various time points during combined milling and enzyme hydrolysis of cotton using an equivalent volume (35%) of bamboo pieces, wood chips or comparative (stainless steel) milling particles.

TABLE 6
Percent conversion of cellulose using an
equivalent volume of milling particles

| Time, | No milling | 1.3 cm SS | Wood | Bamboo |
|---|---|---|---|---|
| h | particles | balls | chips | pieces |
| 2 | 0.2 | 7.5 | 3.4 | 4.5 |
| 4 | 1.1 | 10.7 | 8.7 | 8.1 |
| 6 | 1.7 | 15.3 | 11.7 | 12.1 |
| 24 | 7.0 | 25.5 | 34.0 | 32.7 |

This shows superior conversion of cellulose to reducing sugars after 24 hours from the use of the bamboo pieces as milling particles compared to an equivalent volume of stainless steel balls, and that the performance is similar to wood chips.

1. A process for the enzymatic degradation of a cellulosic substrate, the process comprising agitating a composition with milling particles, wherein the milling particles are or comprise a lignocellulosic material and wherein the composition comprises:
a. the cellulosic substrate;
b. a cellulase enzyme; and
c. a liquid medium.

2. A process according to claim 1, wherein the milling particles are or comprise any one of: wood, nutshells, husks, corn cobs, bamboos, fruit stones or any combination thereof.

3. A process according to claim 1, wherein the milling particles have a density of from 300 Kg/m³ to 1400 Kg/m³.

4. A process according to claim 1, wherein the lignocellulosic material has a lignin content from 15 wt. % to 40 wt. %.

5. A process according to claim 2, wherein the milling particles are or comprise wood and the wood has a Janka hardness greater than 1500 N.

6. A process according to claim 2, wherein the milling particles are or comprise wood, and the wood is optionally unrefined.

7. A process according to claim 1, wherein the milling particles have a size of from 1 mm to 400 mm.

8. A process according to claim 1, wherein the milling particles have an angular shape.

9. A process according to claim 1, wherein the milling particles are or comprise wood and the wood comprises pine, eucalyptus, poplar, acacia, rubberwood, willow, elm, birch, maple, walnut, cherry, apple, chestnut, beech, oak, hickory, alder, mesquite, bamboo or any combination thereof.

10. A process according to claim 1, wherein the cellulosic substrate is or comprises a waste feedstock.

11. A process according to claim 1, wherein the cellulosic substrate is or comprises cotton, flax, rayon, bamboo, sugarcane, sisal, abaca, jute, kenaf, banana, capok, coir, pina raffia, ramie, hemp or a mixture thereof.

12. A process according to claim 1, wherein the cellulosic substrate is or comprises paper, cardboard, pulp of a mixture thereof.

13. A process according to claim 1, wherein the cellulosic substrate is or comprises leaves or grasses.

14. A process according claim 1, wherein the cellulosic substrate has been refined prior to use.

15. A process according to claim 1, wherein the liquid medium is aqueous.

16. A process according to claim 15, wherein the liquid medium comprises at least 95 wt. % of water.

17. A process according to claim 1, wherein the agitation is performed for a period of from 6 hours to 72 hours.

18. A process according to claim 1, wherein the composition has a temperature of from 30 to 50° C. during agitation.

19. A process according to claim 1, wherein the composition comprises from 0.001 to 8 wt. % of cellulase enzymes.

20. A process according to claim 1, wherein the composition comprises from 1 to 40 wt. % of cellulosic substrate.

21. A process according to claim 1, wherein the ratio of milling particles to composition is from 1:20 to 1:1 by weight.

22. A process according to claim 1, wherein the cellulase enzyme is or comprises an endo-cellulase.

23. A process according to claim 1, wherein the cellulase enzyme is or comprises an exo-cellulase.

24. A process according to claim 1, comprising separating the milling particles from the composition after agitation.

25. A process according to claim 24, comprising re-using some or all of the milling particles in a subsequent process for the enzymatic degradation of a cellulosic substrate.

26. A process according to claim 25, wherein the re-using of milling particles in a subsequent process is performed with the addition of virgin milling particles in an amount of from 0.1 to 75% by weight of the total milling particle weight.

27. A process according to claim 1, wherein agitation is performed using a rotary drum or a stirrer.

28. A process according to claim 1, wherein the milling particles decrease in dry mass from 0.5% to 4% after agitation with the composition.

29. A process according to claim 1, wherein enzymatic degradation of the cellulosic substrate produces monosaccharides and/or oligosaccharides.

30. A process according to claim 29, comprising the step of separating the monosaccharides and/or oligosaccharides from the composition after agitation.

31. A process for the production of glucose from a cellulosic substrate, comprising the process according to claim 29, and wherein oligosaccharides are produced and wherein the oligosaccharides are converted to glucose by further enzymatic digestion.

32. A process for the production of one or more alcohols from a cellulosic substrate, comprising a process according to claim 31, and wherein the glucose is fermented to produce said one or more alcohols.

33. A process for the production of one or more biogases from a cellulosic substrate, comprising a process according to claim 29, and wherein the monosaccharides and/or oligosaccharides are converted to the one or more biogases by anaerobic digestion.

34. A process for the production of energy from a cellulosic substrate, comprising a process according to claim 32, and wherein the one or more alcohols are combusted to release energy.

35. An apparatus for the enzymatic degradation of a cellulosic substrate, the apparatus comprising a milling vessel charged with a composition and milling particles, wherein the milling particles are or comprise a lignocellulosic material; and wherein the composition comprises:
a. the cellulosic substrate;
b. a cellulase enzyme; and
c. a liquid medium.

36. A process for the production of energy from a cellulosic substrate comprising a process according to claim 33 wherein the one or more biogases are combusted to release energy.
